(19) United States
(12) Patent Application Publication
(10) Pub. No.: US 2024/0180482 A1
(43) Pub. Date: Jun. 6, 2024

(54) SYSTEMS AND METHODS FOR DIGITAL SPEECH-BASED EVALUATION OF COGNITIVE FUNCTION

(71) Applicants: Arizona Board of Regents on Behalf of Arizona State University, Scottsdale, AZ (US); Aural Analytics, Inc., Scottsdale, AZ (US)

(72) Inventors: Gabriela Stegmann, Mesa, AZ (US); Julie Liss, Scottsdale, AZ (US); Visar Berisha, Tempe, AZ (US); Shira Hahn, Tempe, AZ (US)

(21) Appl. No.: 18/553,335

(22) PCT Filed: Mar. 31, 2022
(86) PCT No.: PCT/US2022/022885

§ 371 Date: Sep. 29, 2023

Related U.S. Application Data
(60) Provisional application No. 63/311,830, filed on Feb. 18, 2022.
(60) Provisional application No. 63/169,069, filed on Mar. 31, 2021.

(51) Int. Cl. A61B 5/00; G10L 15/18; G10L 25/66

(52) U.S. Cl. CPC A61B 5/4803; A61B 5/4088; A61B 5/6898; A61B 5/7267; G10L 15/1815; G10L 25/66

(57) ABSTRACT

Disclosed herein are systems and methods for evaluating or analyzing cognitive function or impairment using speech analysis. In some implementations the evaluation of cognitive function comprises a predicted future cognitive function or change in cognitive function. In some implementations the cognitive function is evaluated using a panel or speech features such as a metric of semantic relevance, MATTR, and other relevant features. In another aspect, a machine learning predictive model for evaluating cognitive function based on speech, comprising: receiving input signal comprising speech audio for a plurality of subjects, to detect one or more metrics of speech identifying classifications corresponding to cognitive function and training a model using machine learning based on a training data set comprising the one or more metrics of speech and the classifications identified in the speech audio, thereby generating a machine learning predictive model configured to generate an evaluation of cognitive function based on speech.

### CROSS-REFERENCE

This application claims the benefit of U.S. Provisional Application Ser. No. 63/311,830, filed Feb. 18, 2022, and U.S. Provisional Application Ser. No. 63/169,069, filed Mar. 31, 2021, the contents of which are incorporated by reference herein in their entirety.

### BACKGROUND

Cognitive decline is associated with deficits in attention to tasks and attention to relevant details. Improved methods are needed to more effectively evaluate cognitive function.

### SUMMARY

Disclosed herein are systems and methods for evaluating or analyzing cognitive function or impairment using speech analysis. In some implementations the evaluation of cognitive function comprises a predicted future cognitive function or change in cognitive function. In some implementations the cognitive function is evaluated using a panel or speech features such as a metric of semantic relevance, MATTR, and other relevant features.

Disclosed herein, In some implementations are systems and methods that generate a metric associated with semantic relevance and utilize the metric to evaluate cognitive function. The metric can be referred to as semantic relevance (SemR). The metric can be algorithmically extracted from speech and used as a measure of overlap between the content of an image (e.g., a picture or photograph) and the words obtained from a speech or audio sample used to describe the picture. The extracted metric can be utilized for evaluation of cross-sectional and/or longitudinal clinical outcomes relating to cognitive function such as, for example, classification according to a plurality of cognitive categories. Examples of such categories include Normal Cognition (NC), Early MCI (EMCI), MCI, and Dementia (D).

Disclosed herein, In some implementations are systems and methods that effectively enable remote and unsupervised test administration that perform comparatively to supervised in-person conditions. Neuropsychological testing normally requires an in-person visit with a trained administrator using standard/fixed materials. Speech-based cognitive testing on mobile devices enables more frequent and timely test administration, but head-to-head comparisons of in-person and remote versions of tests are rare. Accordingly, the systems and methods disclosed herein address this challenge by providing a digital speech analysis framework that can be carried out remotely without supervision, thus enabling more frequent evaluations without compromising accuracy.

Disclosed herein, In some implementations are systems and methods for evaluating cognitive function such as performing detection of cognitive decline using speech and/or language data. In some implementations the systems and methods automatically extract speech (audio) and language (transcripts) features from Cookie Theft picture descriptions (BDAE) to develop two classification models separating healthy participants from those with mild cognitive impairment (MCI) and dementia.

Disclosed herein, In some implementations are systems and methods for evaluating future cognitive decline using detected changes in language. Features extracted from speech transcripts elicited from a task such as Cookie Theft picture description can be used to predict a likelihood a currently healthy subject (e.g., subject has currently undetectable cognitive impairment) will develop some degree of cognitive impairment (e.g., MCI) in the future and/or classify the subject into a category corresponding to a degree of cognitive impairment. This approach leverages the language changes that can occur with cognitive decline to allow for predictions of future cognitive impairment in otherwise currently healthy subjects.

In some aspects, disclosed herein is a device for evaluating cognitive function based on speech, the device comprising: audio input circuitry configured to receive an audio signal provided by a subject; signal processing circuitry configured to: receive the input signal; process the input signal to detect one or more metrics of speech of the subject; and analyze the one or more metrics of speech using a speech assessment algorithm to generate an evaluation of a cognitive function of the subject. In some implementations the evaluation of the cognitive function comprises detection or prediction of future cognitive decline. In some implementations the evaluation of the cognitive function comprises a prediction or classification of normal cognition, early mild cognitive impairment, mild cognitive impairment, or dementia. In some implementations the one or more metrics of speech of the subject comprises a metric of semantic relevance, word count, ratio of unique words to total number of words (MATTR), pronoun-to-noun ratio, propositional density, number of pauses during an audio speech recording within the input signal, or any combination thereof. In some implementations the metric of semantic relevance measures a degree of overlap between a content of a picture and a description of the picture detected from the speech in the input signal. In some implementations the signal processing circuitry is further configured to display an output comprising the evaluation. In some implementations the notification element comprises a display. In some implementations the signal processing circuitry is further configured to cause the display to prompt the subject to provide a speech sample from which the input signal is derived. In some implementations the signal processing circuitry is further configured to utilize at least one machine learning classifier to generate the evaluation of the cognitive function of the subject. In some implementations the signal processing circuitry is configured to utilize a plurality of machine learning classifiers comprising a first classifier configured to evaluate the subject for a first cognitive function or condition and a second classifier configured to evaluate the subject for a second cognitive function or condition.

In some aspects, disclosed herein is a computer-implemented method for evaluating cognitive function based on speech, the method comprising: receiving an input signal provided by a subject; processing the input signal to detect one or more metrics of speech of the subject; and analyzing the one or more metrics of speech using a speech assessment algorithm to generate an evaluation of a cognitive function of the subject. In some implementations the evaluation of the cognitive function comprises detection or prediction of future cognitive decline. In some implementations the evaluation of the cognitive function comprises a prediction or classification of normal cognition, early mild cognitive impairment, mild cognitive impairment, or dementia. In some implementations the one or more metrics of speech of the subject comprises a metric of semantic relevance, word count, ratio of unique words to total number of words (MATTR), pronoun-to-noun ratio, propositional density, number of pauses during an audio speech recording within the input signal, or any combination thereof. In some implementations the metric of semantic relevance measures a degree of overlap between a content of a picture and a description of the picture detected from the speech in the input signal. In some implementations the signal processing circuitry is further configured to display an output comprising the evaluation. In some implementations the notification element comprises a display. In some implementations the method further comprises prompting the subject to provide a speech sample from which the input signal is derived. In some implementations the method further comprises utilizing at least one machine learning classifier to generate the evaluation of the cognitive function of the subject. In some implementations the at least one machine learning classifier comprises a first classifier configured to evaluate the subject for a first cognitive function or condition and a second classifier configured to evaluate the subject for a second cognitive function or condition.

In another aspect, disclosed herein is a computer-implemented method for generating a speech assessment algorithm comprising a machine learning predictive model for evaluating cognitive function based on speech, the method comprising: receiving input signal comprising speech audio for a plurality of subjects; processing the input signal to detect one or more metrics of speech in the speech audio for the plurality of subjects; identifying classifications corresponding to cognitive function for the speech audio for the plurality of subjects; and training a model using machine learning based on a training data set comprising the one or more metrics of speech and the classifications identified in the speech audio, thereby generating a machine learning predictive model configured to generate an evaluation of cognitive function based on speech. In some implementations, the evaluation of the cognitive function comprises detection or prediction of future cognitive decline. In some implementations, the evaluation of the cognitive function comprises a prediction or classification of normal cognition, early mild cognitive impairment, mild cognitive impairment, or dementia. In some implementations, the one or more metrics of speech of the subject comprises a metric of semantic relevance, word count, ratio of unique words to total number of words (MATTR), pronoun-to-noun ratio, propositional density, number of pauses during an audio speech recording within the input signal, or any combination thereof. In some implementations, the metric of semantic relevance measures a degree of overlap between a content of a picture and a description of the picture detected from the speech in the input signal. In some implementations, the method further comprises configuring a computing device with executable instructions for analyzing the one or more metrics of speech using the machine learning predictive model to generate an evaluation of a cognitive function of a subject based on the input speech sample. In some implementations, the computing device is configured to display an output comprising the evaluation. In some implementations, the computing device is a desktop computer, a laptop, a smartphone, a tablet, or a smartwatch. In some implementations, the configuring the computing device with executable instructions comprises providing a software application for installation on the computing device. In some implementations, the computing device is a smartphone, a tablet, or a smartwatch; and wherein the software application is a mobile application. In some implementations, the mobile application is configured to prompt the subject to provide the input speech sample. In some implementations, the input speech sample is processed by one or more machine learning models to generate the one or more metrics of speech; wherein the machine learning predictive model is configured to the evaluation of cognitive function as a composite metric based on the one or more metrics of speech.

Disclosed herein are systems, devices, methods, and non-transitory computer readable storage medium for carrying out any of the speech or audio processing and/or analyses of the present disclosure. Any embodiments specifically directed to a system, a device, a method, or a non-transitory computer readable storage medium is also contemplated as being implemented in any alternative configuration such as a system, a device, a method, or a non-transient/non-transitory computer readable storage medium. For example, any method disclosed herein also contemplates a system or device configured to carry out the method.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a schematic diagram depicting a system for assessing parameters of speech resulting from a health or physiological state or change.

FIG. 2 is a flow diagram illustrating a series of audio pre-processing steps, feature extraction, and analysis according to some embodiments of the present disclosure.

FIG. 3 shows a scatterplot showing the algorithmically analyzed vs manually annotated SemR scores. Each point shows each transcript.

FIG. 4 shows an association between SemR and MMSE. The dark blue line is the expected SemR score for each value of MMSE according to the fixed-effects from the mixed-effects model; the blue shade is the confidence band.

FIG. 5 shows the longitudinal trajectories for HCs (FIG. 3a) and cognitively impaired (FIG. 3b) participants according to the GCM for regions with the most data for each group (approximately Q1-Q3). The dark lines are the expected trajectories according to the fixed-effects of the GCMs and the light shades are the confidence bands.

FIG. 6 shows the means (and 1-standard error bars) for the word counts for supervised and unsupervised samples.

FIG. 7 shows the means (and 1-standard error bars) for the semantic relevance scores for supervised and unsupervised samples.

FIG. 8 shows the means (and 1-standard error bars) for the MATTR scores for supervised and unsupervised samples.

FIG. 9 shows the means (and 1-standard error bars) for the pronoun-to-noun ratios for supervised and unsupervised samples.

FIG. 10 shows an ROC curve for the MCI classification model.

FIG. 11 shows an ROC curve for the Dementia classification model.

FIG. 12A is a scatterplot showing the manually-annotated SemR values vs manually-transcribed algorithmically-computed SemR values.

FIG. 12B is a scatterplot showing manually-transcribed algorithmically-computed SemR values vs ASR-transcribed algorithmically-computed SemR values.

FIG. 12C is a scatterplot showing manually-annotated SemR values vs ASR-transcribed algorithmically-computed SemR values.

FIG. 13 is a boxplot of SemR scores for at-home (unsupervised) and in-clinic (supervised) samples.

FIG. 14 is a test-retest reliability plot for SemR.

FIG. 15 is a scatterplot showing the predicted and observed MMSE values.

FIG. 16A is a longitudinal plot showing the SemR values as a function of age for cognitively unimpaired participants. The dark solid lines are based on the fixed effects of the GCM, and the shaded areas show the 95% confidence bands.

FIG. 16B is a longitudinal plot showing the SemR values as a function of age for cognitively unimpaired declining, MCI, and dementia participants. The dark solid lines are based on the fixed effects of the GCM, and the shaded areas show the 95% confidence bands.

### DETAILED DESCRIPTION OF THE INVENTION

Disclosed herein are systems, devices, and methods for evaluating speech to determine cognitive function or changes to cognitive function. The speech evaluation process may be carried out in an automated manner via a speech assessment algorithm. In some cases, a user device such as a smartphone may have an app installed that displays a picture and captures an audio recording of the user's description of the picture. The audio recording may be stored, processed and/or analyzed locally on the device or be uploaded or transmitted for remote analysis by a remote computing device or server (e.g., on the cloud). This enables local speech monitoring and/or analysis to generate a determination of cognitive function or one or more metrics indicative of cognitive function, for example, when network or internet access is unavailable. Alternatively, the audio recording can be uploaded or transmitted for remote analysis which may help ensure the most current algorithm is used for the audio analysis. For example, updates to the speech assessment algorithm on the app may become out of date and result in less accurate analytical results if the user fails to keep the app updated. In some implementations the automated speech assessment algorithm generates a metric of cognitive function or impairment. The metric can include semantic relevance as a measurement of cognitive impairment.

In some cases, a clinician could prescribe the app for use at home prior to a visit to shorten exam time. Accordingly, the systems, devices, methods, and media disclosed herein can provide for monitoring or analysis of a subject's speech prior to a medical appointment. One or more metrics of cognitive function generated by this initial analysis may then be taken into account by the clinician during the subsequent medical evaluation.

In some cases, a clinician could prescribe the app for use at home in between visits to screen to for changes in cognitive ability. For example, the systems, devices, methods, and media disclosed herein may store the calculated metrics of cognitive function or impairment over a period of time that enables the generation of a longitudinal chart showing the timeline of cognitive function or impairment. This enables a clinician to access a higher resolution timeline that is possible only because of the automated and remote nature of the speech analysis. This higher resolution timeline can allow for a clearer picture of the progression of the subject's cognitive function or impairment that would not be possible with regular in-person medical appointments, for example, biweekly appointments. In some cases, the stored metrics that show changes in cognitive function over time that may warrant an in-person appointment. For example, metrics of cognitive function or impairment that exceeds a fixed (e.g., preset) or variable (e.g., calculated as a standard deviation) threshold value may result in a warning or notification (e.g., a message or alert being displayed through the app installed on the device, emailed to the user, or sent as a text message) being provided to the user or subject advising them to seek medical attention or appointment.

In some cases, the systems, devices, methods, and media disclosed herein provide a software app that enables a clinician to provide telemedicine services for evaluating and/or measuring cognitive function or impairment of a subject. Accordingly, the subject or user may utilize the graphic user interface of an app installed on a user device (e.g., smartphone, tablet, or laptop) to schedule an e-visit or consultation with a clinician or healthcare provider. The app may provide an interactive calendar showing availabilities of healthcare providers for the subject and allowing for appointments to be made with a healthcare provider.

In some cases, the systems, devices, methods, and media disclose herein enable a healthcare provider such as a clinician to use the app in-office as part of a mental and cognitive health exam.

The information set forth herein enable those skilled in the art to practice the embodiments and illustrate the best mode of practicing the embodiments. Upon reading the description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

The systems and methods disclosed herein enables identification and/or prediction of physiological changes, states, or conditions associated with speech production such as cognitive function. This improved approach provides more effective and convenient detection of such physiological states and earlier therapeutic intervention. Disclosed herein is a panel of measures for evaluating speech to assess cognitive function. In certain embodiments, such measures are implemented in a mobile application with a user interface, algorithms for processing the speech, visualization to track these changes, or any combination thereof. Speech analysis provides a novel and unobtrusive approach to this detection and tracking since the data can be collected frequently and using a participant's personal electronic device(s) (e.g., smartphone, tablet computer, etc.).

### Systems and Methods for Assessing Speech

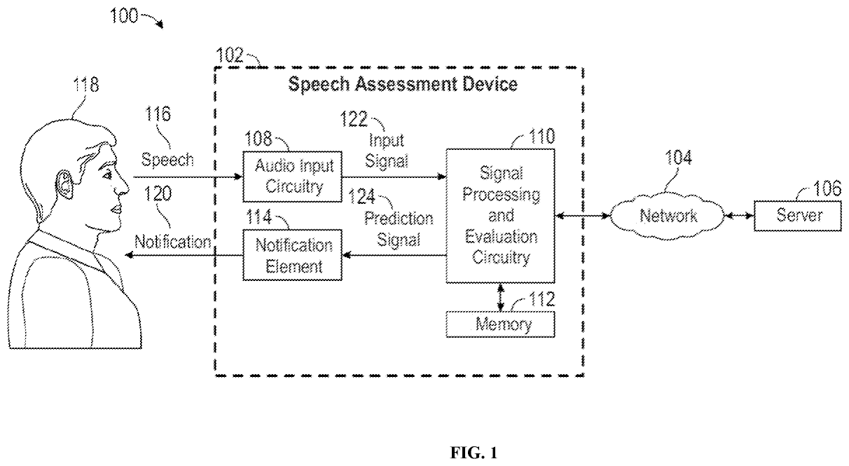

FIG. 1 is a diagram of a system 100 for assessing speech, the system comprising a speech assessment device 102, a network 104, and a server 106. The speech assessment device 102 comprises audio input circuitry 108, signal processing circuitry 110, memory 112, and at least one notification element 114. In certain embodiments, the signal processing circuitry 110 may include, but not necessarily be limited to, audio processing circuitry. In some cases, the signal processing circuitry is configured to provide at least one speech assessment signal (e.g., generated outputs based on algorithmic/model analysis of input feature measurements) based on characteristics of speech provided by a user (e.g., speech or audio stream or data). The audio input circuitry 108, notification element(s) 114, and memory 112 may be coupled with the signal processing circuitry 110 via wired connections, wireless connections, or a combination thereof. The speech assessment device 102 may further comprise a smartphone, a smartwatch, a wearable sensor, a computing device, a headset, a headband, or combinations thereof. The speech assessment device 102 may be configured to receive speech 116 from a user 118 and provide a notification 120 to the user 118 based on processing the speech 116 and any associated signals to assess changes in speech attributable to cognitive function (e.g., cognitive impairment, dementia, etc.). In some cases, the speech assessment device 102 is a computing device accessible by a healthcare professional. For example, a doctor may obtain an audio file of speech provided by a subject and process the audio file using a computing device to determine one or more metrics of cognitive function.

The audio input circuitry 108 may comprise at least one microphone. In certain embodiments, the audio input circuitry 108 may comprise a bone conduction microphone, a near field air conduction microphone array, or a combination thereof. The audio input circuitry 108 may be configured to provide an input signal 122 that is indicative of the speech 116 provided by the user 118 to the signal processing circuitry 110. The input signal 122 may be formatted as a digital signal, an analog signal, or a combination thereof. In certain embodiments, the audio input circuitry 108 may provide the input signal 122 to the signal processing circuitry 110 over a personal area network (PAN). The PAN may comprise Universal Serial Bus (USB), IEEE 1394 (FireWire) Infrared Data Association (IrDA), Bluetooth, ultra-wideband (UWB), Wi-Fi Direct, or a combination thereof. The audio input circuitry 108 may further comprise at least one analog-to-digital converter (ADC) to provide the input signal 122 in digital format.

The signal processing circuitry 110 may comprise a communication interface (not shown) coupled with the network 104 and a processor (e.g., an electrically operated microprocessor (not shown) configured to execute a pre-defined and/or a user-defined machine readable instruction set, such as may be embodied in computer software) configured to receive the input signal 122. The communication interface may comprise circuitry for coupling to the PAN, a local area network (LAN), a wide area network (WAN), or a combination thereof. The processor may be configured to receive instructions (e.g., software, which may be periodically updated) for extracting one or more metrics of speech (e.g., metric of semantic relevance, MATTR, etc.) of the user 118.

Generating an assessment or evaluation of cognitive function of the user 118 can include measuring one or more of the speech features described herein. For example, the speech production features may include one or more of an automated metric of semantic relevance, age, sex or gender, word count, MATTR, pronouns-to-nouns ratio, as described herein above.

Machine learning algorithms or models based on these speech measures may be used assess changes in cognitive function.

In certain embodiments, such machine learning algorithms (or other signal processing approaches) may analyze a panel of multiple speech features extracted from one or more speech audios using one or more algorithms or models to generate an evaluation or assessment of cognitive function. The evaluation can include or incorporate a measure of semantic relevance. In some cases, the evaluation can be based on the measure of semantic relevance alone or in combination with other metrics. For example, a subject may be classified within a particular cognitive category based at least on a measure of semantic relevance generated from audio data collected for the subject.

In certain embodiments, the processor may comprise an ADC to convert the input signal 122 to digital format. In other embodiments, the processor may be configured to receive the input signal 122 from the PAN via the communication interface. The processor may further comprise level detect circuitry, adaptive filter circuitry, voice recognition circuitry, or a combination thereof. The processor may be further configured to process the input signal 122 using one or more metrics or features derived from a speech input signal and produce a speech assessment signal, and provide a cognitive function prediction signal 124 to the notification element 114. The cognitive function signal 124 may be in a digital format, an analog format, or a combination thereof. In certain embodiments, the cognitive function signal 124 may comprise one or more of an audible signal, a visual signal, a vibratory signal, or another user-perceptible signal. In certain embodiments, the processor may additionally or alternatively provide the cognitive function signal 124 (e.g., predicted cognitive function or classification or predicted future change in cognitive function) over the network 104 via a communication interface.

The processor may be further configured to generate a record indicative of the cognitive function signal 124. The record may comprise a sample identifier and/or an audio segment indicative of the speech 116 provided by the user 118. In certain embodiments, the user 118 may be prompted to provide current symptoms or other information about their current well-being to the speech assessment device 102 for assessing speech production and associated cognitive function. Such information may be included in the record, and may further be used to aid in identification or further prediction of changes in cognitive function.

The record may further comprise a location identifier, a time stamp, a physiological sensor signal (e.g., heart rate, blood pressure, temperature, or the like), or a combination thereof being correlated to and/or contemporaneous with the speech signal 124. The location identifier may comprise a Global Positioning System (GPS) coordinate, a street address, a contact name, a point of interest, or a combination thereof. In certain embodiments, a contact name may be derived from the GPS coordinate and a contact list associated with the user 118. The point of interest may be derived from the GPS coordinate and a database including a plurality of points of interest. In certain embodiments, the location identifier may be a filtered location for maintaining the privacy of the user 118. For example, the filtered location may be “user's home”, “contact's home”, “vehicle in transit”, “restaurant”, or “user's work”. In certain embodiments, the record may include a location type, wherein the location identifier is formatted according to the location type.

The processor may be further configured to store the record in the memory 112. The memory 112 may be a non-volatile memory, a volatile memory, or a combination thereof. The memory 112 may be wired to the signal processing circuitry 110 using an address/data bus. In certain embodiments, the memory 112 may be portable memory coupled with the processor.

In certain embodiments, the processor may be further configured to send the record to the network 104, wherein the network 104 sends the record to the server 106. In certain embodiments, the processor may be further configured to append to the record a device identifier, a user identifier, or a combination thereof. The device identifier may be unique to the speech assessment device 102. The user identifier may be unique to the user 118. The device identifier and the user identifier may be useful to a medical treatment professional and/or researcher, wherein the user 118 may be a patient of the medical treatment professional. A plurality of records for a user or subject may be stored locally on a user computing device (e.g., a smartphone or tablet) and/or remotely over a remote computing device (e.g., a cloud server maintained by or for a healthcare provider such as a hospital). The records can be processed to generate information that may be useful to the subject or healthcare provider, for example, a timeline of one or more metrics of cognitive function or impairment generated from a plurality of speech audio files or samples collected repeatedly across multiple time points. This information may be presented to a user or healthcare provider on a graphic user interface of the computing device.

The network 104 may comprise a PAN, a LAN, a WAN, or a combination thereof. The PAN may comprise USB, IEEE 1394 (FireWire) IrDA, Bluetooth, UWB, Wi-Fi Direct, or a combination thereof. The LAN may include Ethernet, 802.11 WLAN, or a combination thereof. The network 104 may also include the Internet.

The server 106 may comprise a personal computer (PC), a local server connected to the LAN, a remote server connected to the WAN, or a combination thereof. In certain embodiments, the server 106 may be a software-based virtualized server running on a plurality of servers.

In certain embodiments, at least some signal processing tasks may be performed via one or more remote devices (e.g., the server 106) over the network 104 instead of within a speech assessment device 102 that houses the audio input circuitry 108.

In certain embodiments, a speech assessment device 102 may be embodied in a mobile application configured to run on a mobile computing device (e.g., smartphone, smartwatch) or other computing device. With a mobile application, speech samples can be collected remotely from patients and analyzed without requiring patients to visit a clinic. A user 118 may be periodically queried (e.g., two, three, four, five, or more times per day) to provide a speech sample. For example, the notification element 114 may be used to prompt the user 118 to provide speech 116 from which the input signal 122 is derived, such as through a display message or an audio alert. The notification element 114 may further provide instructions to the user 118 for providing the speech 116 (e.g., displaying a passage for the user 118 to read). In certain embodiments, the notification element 114 may request current symptoms or other information about the current well-being of the user 118 to provide additional data for analyzing the speech 116.

In certain embodiments, a notification element may include a display (e.g., LCD display) that displays text and prompts the user to read the text. Each time the user provides a new sample using the mobile application, one or more metrics of the user's speech abilities indicative of cognitive function or impairment may be automatically extracted (e.g., metrics of semantic relevance, MATTR, pronoun-to-noun ratio, etc.). One or more machine-learning algorithms based on these metrics or features may be implemented to aid in identifying and/or predicting a cognitive function or condition of the user that is associated with the speech capabilities. In some cases, a composite metric may be generated utilizing a plurality of the metrics of speech abilities. For example, a composite metric for overall cognitive function may be generated according to a machine learning model configured to output the composite metric based on input data comprising semantic relevance, MATTR, pronoun-to-noun ratio, and/or other metrics disclosed herein.

In certain embodiments, a user may download a mobile application to a personal computing device (e.g., smartphone), optionally sign in to the application, and follow the prompts on a display screen. Once recording has finished, the audio data may be automatically uploaded to a secure server (e.g., a cloud server or a traditional server) where the signal processing and machine learning algorithms operate on the recordings.

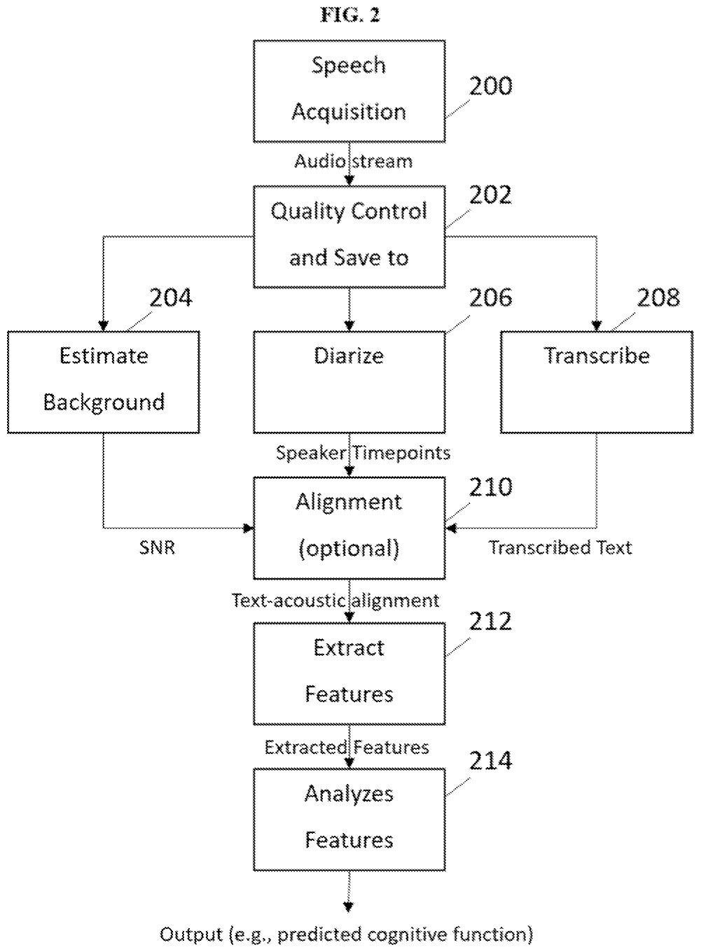

FIG. 2 is a flow diagram illustrating a process for extracting features of speech for evaluating cognitive function such as, for example, dementia or mild cognitive impairment (MCI). As shown in FIG. 2, the process for speech/language feature extraction and analysis can include one or more steps such as speech acquisition 200, quality control 202, background noise estimation 204, diarization 206, transcription 208, optional alignment 210, feature extraction 212, and/or feature analysis 214. In some implementations the systems, devices, and methods disclosed herein include a speech acquisition step. Speech acquisition 200 can be performed using any number of audio collection devices. Examples include microphones or audio input devices on a laptop or desktop computer, a portable computing device such as a tablet, mobile devices (e.g., smartphones), digital voice recorders, audiovisual recording devices (e.g., video camera), and other suitable devices. In some implementations the speech or audio is acquired through passive collection techniques. For example, a device may be passively collecting background speech via a microphone without actively eliciting the speech from a user or individual. The device or software application implemented on the device may be configured to begin passive collection upon detection of background speech. Alternatively, or in combination, speech acquisition can include active elicitation of speech. For example, a mobile application implemented on the device may include instructions prompting speech by a user or individual. In some implementations the user is prompted to provide a verbal description such as, for example, a picture description. As an illustrative example, the picture description can be according to a Cookie Theft picture description task. Other audio tasks can be provided to avoid skewing of the speech analysis results due to user familiarization with the task. For example, the mobile application may include a rotating set of audio tasks such that a user may be prompted to perform a first audio task (e.g., Cookie Theft picture description) during a first audio collection session, a second audio task during a second audio collection session at a later time, and so on until the schedule of audio tasks has been completed. In some cases, the mobile application is updated with new audio tasks, for example, when the user has used a certain number or proportion of the current audio tasks or has exhausted the current audio tasks. These features enable the frequent monitoring of user speech abilities and/or cognitive function or impairment over time, which can be especially valuable for detecting significant changes in a person suffering from a disease or disorder affecting cognitive function (e.g., dementia).

In some implementations the systems, devices, and methods disclosed herein utilize a dialog bot or chat bot that is configured to engage the user or individual in order to elicit speech. As an illustrative example, the bot may engage in a conversation with the user (e.g., via a graphic user interface such as a smartphone touchscreen or via an audio dialogue). Alternatively or in combination with a conversation, the bot may simply provide instructions to the user to perform a particular task (e.g., instructions to vocalize pre-written speech or sounds). In some cases, the speech or audio is not limited to spoken words, but can include nonverbal audio vocalizations made by the user or individual. For example, the user may be prompted with instructions to make a sound that is not a word for a certain duration.

In some implementations the systems, devices, and methods disclosed herein include a quality control step 202. The quality control step may include an evaluation or quality control checkpoint of the speech or audio quality. Quality constraints may be applied to speech or audio samples to determine whether they pass the quality control checkpoint. Examples of quality constraints include (but are not limited to) signal to noise ratio (SNR), speech content (e.g., whether the content of the speech matches up to a task the user was instructed to perform), audio signal quality suitability for downstream processing tasks (e.g., speech recognition, diarization, etc.). Speech or audio data that fails this quality control assessment may be rejected, and the user asked to repeat or redo an instructed task (or alternatively, continue passive collection of audio/speech). Speech or audio data that passes the quality control assessment or checkpoint may be saved on the local device (e.g., user smartphone, tablet, or computer) and/or on the cloud. In some cases, the data is both saved locally and backed up on the cloud. In some implementations one or more of the audio processing and/or analysis steps are performed locally or remotely on the cloud.

In some implementations the systems, devices, and methods disclosed herein include background noise estimation 204. Background noise estimation can include metrics such as a signal-to-noise ratio (SNR). SNR is a comparison of the amount of signal to the amount background noise, for example, ratio of the signal power to the noise power in decibels. Various algorithms can be used to determine SNR or background noise with non-limiting examples including data-aimed maximum-likelihood (ML) signal-to-noise ratio (SNR) estimation algorithm (DAML), decision-directed ML SNR estimation algorithm (DDML) and an iterative ML SNR estimation algorithm.

In some implementations the systems, devices, and methods disclosed herein perform audio analysis of speech/audio data stream such as speech diarization 206 and speech transcription 208. The diarization process can include speech segmentation, classification, and clustering. In some cases when there is only one speaker, diarization is optional. The speech or audio analysis can be performed using speech recognition and/or speaker diarization algorithms. Speaker diarization is the process of segmenting or partitioning the audio stream based on the speaker's identity. As an example, this process can be especially important when multiple speakers are engaged in a conversation that is passively picked up by a suitable audio detection/recording device. In some implementations the diarization algorithm detects changes in the audio (e.g., acoustic spectrum) to determine changes in the speaker, and/or identifies the specific speakers during the conversation. An algorithm may be configured to detect the change in speaker, which can rely on various features corresponding to acoustic differences between individuals. The speaker change detection algorithm may partition the speech/audio stream into segments. These partitioned segments may then be analyzed using a model configured to map segments to the appropriate speaker. The model can be a machine learning model such as a deep learning neural network. Once the segments have been mapped (e.g., mapping to an embedding vector), clustering can be performed on the segments so that they are grouped together with the appropriate speaker(s).

Techniques for diarization include using a Gaussian mixture model, which can enable modeling of individual speakers that allows frames of the audio to be assigned (e.g., using Hidden Markov Model). The audio can be clustered using various approaches. In some implementations the algorithm partitions or segments the full audio content into successive clusters and progressively attempts to combine the redundant clusters until eventually the combined cluster corresponds to a particular speaker. In some implementations algorithm begins with a single cluster of all the audio data and repeatedly attempts to split the cluster until the number of clusters that has been generated is equivalent to the number of individual speakers. Machine learning approaches are applicable to diarization such as neural network modeling. In some implementations a recurrent neural network transducer (RNN-T) is used to provide enhanced performance when integrating both acoustic and linguistic cues. Examples of diarization algorithms are publicly available (e.g., Google).

Speech recognition (e.g., transcription of the audio/speech) may be performed sequentially or together with the diarization. The speech transcript and diarization can be combined to generate an alignment of the speech to the acoustics (and/or speaker identity). In some cases, passive and active speech are evaluated using different algorithms. Standard algorithms that are publicly available and/or open source may be used for passive speech diarization and speech recognition (e.g., Google and Amazon open source algorithms may be used). Non-algorithmic approaches can include manual diarization. In some implementations diarization and transcription are not required for certain tasks. For example, the user or individual may be instructed or required to perform certain tasks such as sentence reading tasks or sustained phonation tasks in which the user is supposed to read a pre-drafted sentence(s) or to maintain a sound for an extended period of time. In such tasks, transcription may not be required because the user is being instructed on what to say. Alternatively, certain actively acquired audio may be analyzed using standard (e.g., non-customized) algorithms or, in some cases, customized algorithms to perform diarization and/or transcription. In some implementations the dialogue or chat bot is configured with algorithm(s) to automatically perform diarization and/or speech transcription while interacting with the user

In some implementations the speech or audio analysis comprises alignment 210 of the diarization and transcription outputs. The performance of this alignment step may depend on the downstream features that need to be extracted. For example, certain features require the alignment to allow for successful extraction (e.g., features based on speaker identity and what the speaker said), while others do not. In some implementations the alignment step comprises using the diarization output to extract the speech from the speaker of interest. Standard algorithms may be used with non-limiting examples including Kaldi, gentle, Montreal forced aligner), or customized alignment algorithms (e.g., using algorithms trained with proprietary data).

In some implementations the systems, devices, and methods disclosed herein perform feature extraction 212 from one or more of the SNR, diarization, and transcription outputs. One or more extracted features can be analyzed 214 to predict or determine an output comprising one or more composites or related indicators of speech production. In some implementations the output comprises an indicator of a physiological condition such as a cognitive status or impairment (e.g., dementia-related cognitive decline).

The systems, devices, and methods disclosed herein may implement or utilize a plurality or chain or sequence of models or algorithms for performing analysis of the features extracted from a speech or audio signal. In some implementations the plurality of models comprises multiple models individually configured to generate specific composites or perceptual dimensions. In some implementations one or more outputs of one or more models serve as input for one or more next models in a sequence or chain of models. In some implementations one or more features and/or one or more composites are evaluated together to generate an output. In some implementations a machine learning algorithm or ML-trained model (or other algorithm) is used to analyze a plurality of feature or feature measurements/metrics extracted from the speech or audio signal to generate an output such as a composite. In some implementations the systems, devices, and methods disclosed herein combine the features to produce one or more composites that describe or correspond to an outcome, estimation, or prediction.

In some implementations the systems, devices, and methods disclosed herein utilize one or more metrics of speech for evaluating cognitive function. One example is a metric of semantic relevance. A metric of semantic relevance can be generated using one or more content information units. As an illustrative example, a complex picture is shown to the participants, and the participants describe the picture. The “content information units” are the aspects of the picture that should be described. For instance, the Boston Diagnostic Aphasia Examination shows a picture where a boy is stealing cookies while the mother is distracted. Examples of “content information units” are “boy”, “cookies”, “stealing”, and “distracted.” The relevant components of the picture (objects, actions, places, and inferences) are the “content information units.” In some implementations such content information units are extracted from digital speech using a text processing algorithm.

In some implementations semantic relevance is a text processing algorithm that operates on a transcript of the response to a picture description. It is configured to assess the number of relevant words relative to the number of irrelevant words in a transcript. The algorithm scans the transcript of the speech, looking for evidence of each possible content information unit. The input to the algorithm is a transcript of the speech, and a list of possible content information units (boy, stealing cookies, etc.). A family of words, defined by previously collected transcripts and from word similarity metrics, is generated for each content unit. Each word in the transcript that matches one of the content-unit-related families of words is considered ‘relevant’.

In some implementations an evaluation of mild cognitive impairment accurately correlates to a reference standard such as the MMSE, which is an exam used by clinicians to measure cognitive impairment. The exam asks patients to answer questions to assess orientation (what the current day of the week is, where the patient is), memory, language processing (spelling, word finding, articulation, writing), drawing ability, and ability to follow instructions. Scores range from 0 to 30, where high scores indicate healthy cognition, and low scores indicate impaired cognition.

In some implementations one or more metrics are used to evaluate cognitive function. Examples of such metrics include semantic relevance, MATTR (a measure of the total number of unique words relative to the total number words), word count (a count of the number of words in the transcript), pronoun to noun ratio (the number of pronouns in the transcript relative to the number of nouns), and propositional density (a measure of the complexity of the grammar used in each sentence). In some implementations a first model configured to measure or detect mild cognitive impairment utilizes metrics including one or more of MATTR, pronoun-to-noun ratio, or propositional density. In some implementations a second model configured to measure or detect dementia utilizes metrics including one or more of parse tree height (another measure of the complexity of grammar in each sentence), mean length of word (uses the transcript to count the number of letters in each word), type to token ratio (similar to MATTR), proportion of details identified correctly (measures how many of the “content units” associated with the displayed picture are mentioned; e.g., a score of 50% means that a participant named half of the expected content units), or duration of pauses relative to speaking duration (a proxy for pauses to search for words or thoughts during language processing). The duration of pauses relative to speaking duration can be obtained using a signal processing algorithm to determine what parts of the recording contain speech and which contain non-speech or silence. The value is a ratio of the amount of speech relative to the amount of non-speech.

In some implementations the evaluation of cognitive function is generated at a high accuracy. The accuracy of the evaluation or output of the speech assessment algorithm can be evaluated against independent samples (e.g., at least 100 samples) that form a validation or testing data set not used for training the machine learning model. In some implementations the evaluation has an AUC ROC of at least 0.70, at least 0.75, or at least 0.80. In some implementations the evaluation has a sensitivity of at least 0.70, at least 0.75, or at least 0.80 and/or a specificity of at least 0.70, at least 0.75, or at least 0.80.

### System and Device Interfaces

In some implementations the systems, devices, and methods disclosed herein comprise a user interface for prompting or obtaining an input speech or audio signal, and delivering the output or notification to the user. The user interface may be communicatively coupled to or otherwise in communication with the audio input circuitry 108 and/or notification element 114 of the speech assessment device 102. The speech assessment device can be any suitable electronic device capable of receiving audio input, processing/analyzing the audio, and providing the output signal or notification. Non-limiting examples of the speech assessment device include smartphones, tablets, laptops, desktop computers, and other suitable computing devices.

In some implementations the interface comprises a touchscreen for receiving user input and/or displaying an output or notification associated with the output. In some cases, the output or notification is provided through a non-visual output element such as, for example, audio via a speaker. The audio processing and analytics portions of the instant disclosure are provided via computer software or executable instructions. In some implementations the computer software or executable instructions comprise a computer program, a mobile application, or a web application or portal. The computer software can provide a graphic user interface via the device display. The graphic user interface can include a user login portal with various options such as to input or upload speech/audio data/signal/file, review current and/or historical speech/audio inputs and outputs (e.g., analyses), and/or send/receive communications including the speech/audio inputs or outputs.

In some implementations the user is able to configure the software based on a desired physiological status the user wants to evaluate or monitor (e.g., cognitive function). In some implementations the graphic user interface provides graphs, charts, and other visual indicators for displaying the status or progress of the user with respect to the physiological status or condition, for example, cognitive impairment or dementia.

In some implementations the computer software is a mobile application and the device is a smartphone. This enables a convenient, portable mechanism to monitor cognitive function or status based on speech analysis without requiring the user to be in the clinical setting. In some implementations the mobile application includes a graphic user interface allowing the user to login to an account, review current and historical speech and/or cognitive function analysis results, and visualize the results over time.

In some implementations the device and/or software is configured to securely transmit the results of the speech analysis to a third party (e.g., healthcare provider of the user). In some implementations the user interface is configured to provide performance metrics associated with the physiological or health condition (e.g., cognitive function).

### Machine Learning Algorithms

In some implementations the systems, devices, and methods disclosed herein utilize one or algorithms or models configured to evaluate or assess speech metrics or features extracted from digital speech audio to generate a prediction or determination regarding cognitive function. In some cases, one or more algorithms are used to process raw speech or audio data (e.g., diarization). The algorithm(s) used for speech processing may include machine learning and non-machine learning algorithms. The extracted feature(s) may be input into an algorithm or ML-trained model to generate an output. In some implementations one or more features, one or more composites, or a combination of one or more features and one or more composites are provided as input to a machine learning algorithm or ML-trained model to generate the desired output.

In some implementations the signal processing and evaluation circuitry comprises one or more machine learning modules comprising machine learning algorithms or ML-trained models for evaluating the speech or audio signal, the processed signal, the extracted features, or the extracted composite(s) or a combination of features and composite(s). A machine learning module may be trained on one or more training data sets. A machine learning module may include a model trained on at least about: 1, 2, 3, 4, 5, 6, 7, 8, 9, 10, 20, 30, 40, 50, 60, 70, 80, 90, 100 data sets or more (e.g., speech/audio signals). A machine learning module may be validated with one or more validation data sets. A validation data set may be independent from a training data set. The machine learning module(s) and/or algorithms/models disclosed herein can be implemented using computing devices or digital process devices or processors as disclosed herein.

A machine learning algorithm may use a supervised learning approach. In supervised learning, the algorithm can generate a function or model from training data. The training data can be labeled. The training data may include metadata associated therewith. Each training example of the training data may be a pair consisting of at least an input object and a desired output value (e.g., a score or classification). A supervised learning algorithm may require the individual to determine one or more control parameters. These parameters can be adjusted by optimizing performance on a subset, for example a validation set, of the training data. After parameter adjustment and learning, the performance of the resulting function/model can be measured on a test set that may be separate from the training set. Regression methods can be used in supervised learning approaches.

A machine learning algorithm may use an unsupervised learning approach. In unsupervised learning, the algorithm may generate a function/model to describe hidden structures from unlabeled data (e.g., a classification or categorization that cannot be directly observed or computed). Since the examples given to the learner are unlabeled, there is no evaluation of the accuracy of the structure that is output by the relevant algorithm. Approaches to unsupervised learning include clustering, anomaly detection, and neural networks.

A machine learning algorithm is applied to patient data to generate a prediction model. In some implementations a machine learning algorithm or model may be trained periodically. In some implementations a machine learning algorithm or model may be trained non-periodically.

As used herein, a machine learning algorithm may include learning a function or a model. The mathematical expression of the function or model may or may not be directly computable or observable. The function or model may include one or more parameter(s) used within a model. In some implementations a machine learning algorithm comprises a supervised or unsupervised learning method such as, for example, support vector machine (SVM), random forests, gradient boosting, logistic regression, decision trees, clustering algorithms, hierarchical clustering, K-means clustering, or principal component analysis. Machine learning algorithms may include linear regression models, logistical regression models, linear discriminate analysis, classification or regression trees, naive Bayes, K-nearest neighbor, learning vector quantization (LVQ), support vector machines (SVM), bagging and random forest, boosting and Adaboost machines, or any combination thereof. In some implementations machine learning algorithms include artificial neural networks with non-limiting examples of neural network algorithms including perceptron, multilayer perceptrons, back-propagation, stochastic gradient descent, Hopfield network, and radial basis function network. In some implementations the machine learning algorithm is a deep learning neural network. Examples of deep learning algorithms include convolutional neural networks (CNN), recurrent neural networks, and long short-term memory networks.

### Digital Processing Device

The systems, devices, and methods disclosed herein may be implemented using a digital processing device that includes one or more hardware central processing units (CPUs) or general purpose graphics processing units (GPGPUs) that carry out the device's functions. The digital processing device further comprises an operating system configured to perform executable instructions. The digital processing device is optionally connected to a computer network. The digital processing device is optionally connected to the Internet such that it accesses the World Wide Web. The digital processing device is optionally connected to a cloud computing infrastructure. Suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein.

Typically, a digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some implementations the operating system is provided by cloud computing.

A digital processing device as described herein either includes or is operatively coupled to a storage and/or memory device. The storage and/or memory device is one or more physical apparatuses used to store data or programs on a temporary or permanent basis. In some implementations the device is volatile memory and requires power to maintain stored information. In some implementations the device is non-volatile memory and retains stored information when the digital processing device is not powered. In further embodiments, the non-volatile memory comprises flash memory. In some implementations the non-volatile memory comprises dynamic random-access memory (DRAM). In some implementations the non-volatile memory comprises ferroelectric random access memory (FRAM). In some implementations the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

A system or method as described herein can be used to generate, determine, and/or deliver an evaluation of speech abilities and/or cognitive function or impairment which may optionally be used to determine whether a subject falls within at least one of a plurality of classifications (e.g., no cognitive impairment, mild cognitive impairment, moderate cognitive impairment, severe cognitive impairment). In addition, In some implementations a system or method as described herein generates a database as containing or comprising one or more records or user data such as captured speech samples and/or evaluations or outputs generated by a speech assessment algorithm. In some implementations a database herein provides a collection of records that may include speech audio files or samples, timestamps, geolocation information, and other metadata.

Some embodiments of the systems described herein are computer based systems. These embodiments include a CPU including a processor and memory which may be in the form of a non-transitory computer-readable storage medium. These system embodiments further include software that is typically stored in memory (such as in the form of a non-transitory computer-readable storage medium) where the software is configured to cause the processor to carry out a function. Software embodiments incorporated into the systems described herein contain one or more modules.

In various embodiments, an apparatus comprises a computing device or component such as a digital processing device. In some of the embodiments described herein, a digital processing device includes a display to send visual information to a user. Non-limiting examples of displays suitable for use with the systems and methods described herein include a liquid crystal display (LCD), a thin film transistor liquid crystal display (TFT-LCD), an organic light emitting diode (OLED) display, an OLED display, an active-matrix OLED (AMOLED) display, or a plasma display.

A digital processing device, in some of the embodiments described herein includes an input device to receive information from a user. Non-limiting examples of input devices suitable for use with the systems and methods described herein include a keyboard, a mouse, trackball, track pad, or stylus. In some implementations the input device is a touch screen or a multi-touch screen.

### Non-Transitory Computer-Readable Storage Medium

The systems and methods described herein typically include one or more non-transitory (non-transient) computer-readable storage media encoded with a program including instructions executable by the operating system of an optionally networked digital processing device. In some embodiments of the systems and methods described herein, the non-transitory storage medium is a component of a digital processing device that is a component of a system or is utilized in a method. In still further embodiments, a computer-readable storage medium is optionally removable from a digital processing device. In some implementations a computer-readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Programs

Typically the systems and methods described herein include at least one computer program, or use of the same. A computer program includes a sequence of instructions, executable in the digital processing device's CPU, written to perform a specified task. Computer-readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages. The functionality of the computer-readable instructions may be combined or distributed as desired in various environments. In some implementations a computer program comprises one sequence of instructions. In some implementations a computer program comprises a plurality of sequences of instructions. In some implementations a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some implementations software modules are in one computer program or application. In other embodiments, software modules are in more than one computer program or application. In some implementations software modules are hosted on one machine. In other embodiments, software modules are hosted on more than one machine. In further embodiments, software modules are hosted on cloud computing platforms. In some implementations software modules are hosted on one or more machines in one location. In other embodiments, software modules are hosted on one or more machines in more than one location.

### Mobile Application

In some implementations a computer program includes a mobile application provided to a mobile electronic device. In some implementations the mobile application is provided to a mobile electronic device at the time it is manufactured. In other embodiments, the mobile application is provided to a mobile electronic device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some implementations a computer program includes a standalone application, which is a program that is run as an independent computer process, not an add-on to an existing process, e.g. not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some implementations a computer program includes one or more executable compiled applications.

### Software Module

In some implementations the platforms, media, methods and applications described herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some implementations software modules are in one computer program or application. In other embodiments, software modules are in more than one computer program or application. In some implementations software modules are hosted on one machine. In other embodiments, software modules are hosted on more than one machine. In further embodiments, software modules are hosted on cloud computing platforms. In some implementations software modules are hosted on one or more machines in one location. In other embodiments, software modules are hosted on one or more machines in more than one location.

### Databases

Typically, the systems and methods described herein include and/or utilize one or more databases. In view of the disclosure provided herein, those of skill in the art will recognize that many databases are suitable for storage and retrieval of baseline datasets, files, file systems, objects, systems of objects, as well as data structures and other types of information described herein. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some implementations a database is internet-based. In further embodiments, a database is web-based. In still further embodiments, a database is cloud computing-based. In other embodiments, a database is based on one or more local computer storage devices.

Although the detailed description contains many specifics, these should not be construed as limiting the scope of the disclosure but merely as illustrating different examples and aspects of the present disclosure. It should be appreciated that the scope of the disclosure includes other embodiments not discussed in detail above. Various other modifications, changes and variations which will be apparent to those skilled in the art may be made in the arrangement, operation and details of the method and apparatus of the present disclosure provided herein without departing from the spirit and scope of the invention as described herein. For example, one or more aspects, components or methods of each of the examples as disclosed herein can be combined with others as described herein, and such modifications will be readily apparent to a person of ordinary skill in the art. For each of the methods disclosed herein, a person of ordinary skill in the art will recognize many variations based on the teachings described herein. The steps may be completed in a different order. Steps may be added or deleted. Some of the steps may comprise sub-steps of other steps. Many of the steps may be repeated as often as desired, and the steps of the methods can be combined with each other.

### EXAMPLES

### Example 1: Semantic Relevance

Data from the Dementia Bank and Wisconsin Registry for Alzheimer's Prevention (WRAP) were combined, and participants (average age 63.7) included: NC (N=918; 647 F), EMCI (n=180; 110 F), MCI (n=26, 9 F), and D (n=195, 126 F). Participants provided Cookie Theft descriptions and Mini-Mental State Exam (MMSE) assessments on an average of 2.1 occasions/participant, assessed on average 2.4 years apart (total n=2,717). A metric of semantic relevance (SemR) was algorithmically computed from each picture description transcript. Transcripts were also hand-coded for “content information units” as a ground-truth comparison with automated SemR. Cross-sectionally, a mixed-effects model was used to calculate relationships between SemR and ground truth, and between SemR and MMSE. Within-speaker SemR longitudinal trajectories were estimated using growth curve models (GCM) for each group.

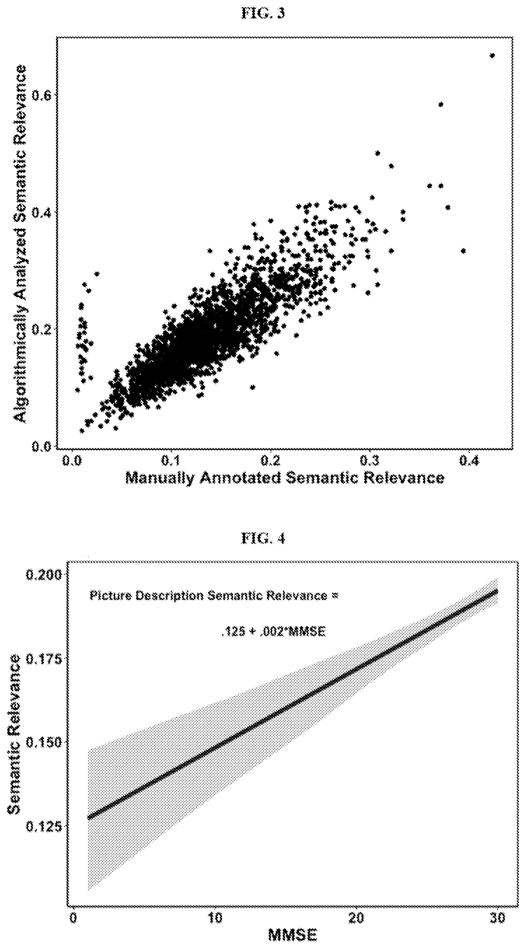

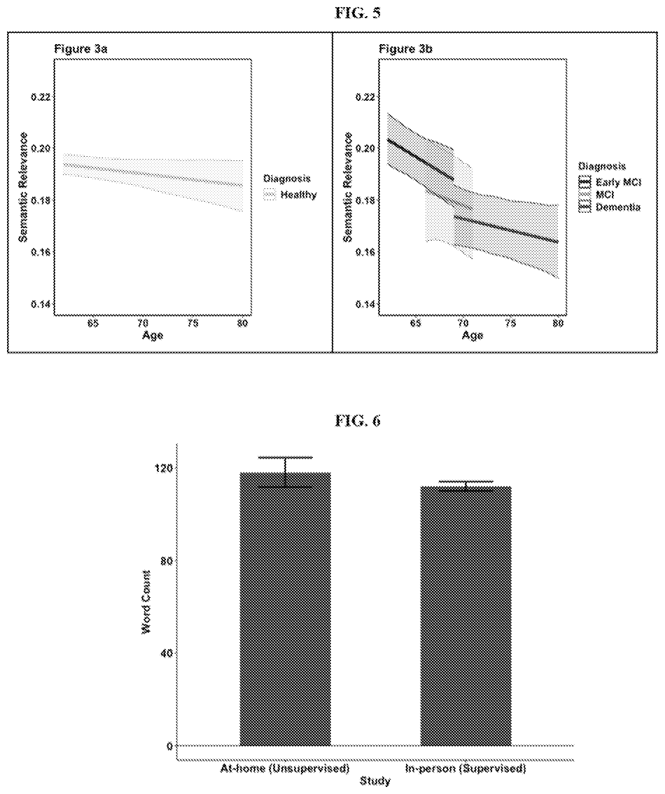

FIG. 3 shows strong correlation between automatic and hand-coded SemR (r=0.85, p<0.05). FIG. 4 shows SemR was significantly related to MMSE (b=0.002, p<0.05), such that decrease in MMSE resulted in decrease in SemR. FIG. 5 shows longitudinal GCMs showing that SemR declined with age for all groups. The decline was slowest for NCs, steepened for the EMCI and MCI groups, and then slowed again for D, who had the lowest scores. FIG. 5 displays SemR trajectories and confidence bands for age ranges with the most data for each group. SemR has a standard error of measurement (SEM) of 0.05.

The results show that SemR is reliable, shows convergent validity with MMSE, and correlates strongly with manual hand-counts. The data confirms that SemR declines with age and severity of cognitive impairment, with the speed of decline differing by level of impairment.

### Example 2: Comparison of Remote and In-Person Digital Speech-Based Measures of Cognition

Two data sets containing Cookie Theft picture descriptions (BDAE) were obtained, one collected in-person, under supervision (Wisconsin Registry for Alzheimer's Prevention (WRAP); N=912; age=62.2 (SD=6.7), 70% F) and one collected remotely, without supervision (participants online; N=93, age=36.5 (SD=11.5), 65% F). WRAP participants were judged to be cognitively normal and non-declining, and online participants self-reported as healthy. Each participant provided one picture description, yielding 93 remote and 912 in-person transcribed descriptions. Language metrics previously used in dementia research were extracted: word count (number of words spoken), MATTR (ratio of unique words to total number of words), pronoun-to-noun ratio, and semantic relevance. Comparing MATTR, pronoun-to-noun ratio, and semantic relevance values elicited in-person and remotely is important because these three characteristics have been found to be impacted by declines in verbal learning and memory. Differences in word counts may reflect different levels of motivation in the two settings.

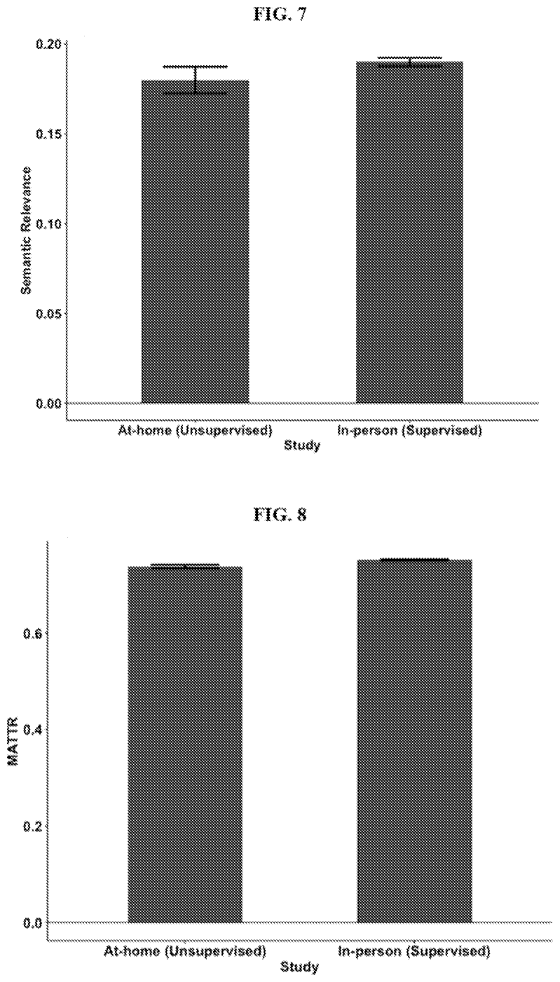

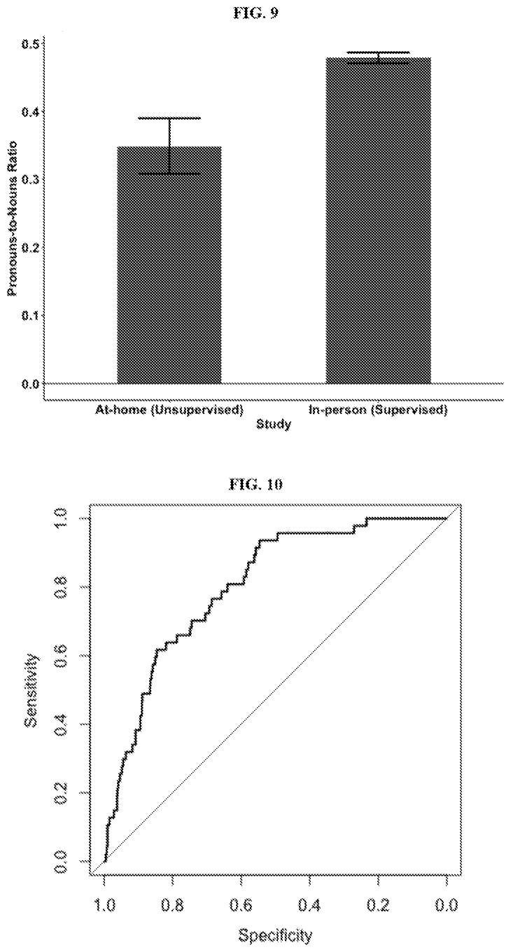

Using Cohen's d effect sizes, differences in mean word count between in-person and remote participant transcripts was negligible (112 words in-person and 118 words remote; d=0.10, p=0.21) as shown in FIG. 6, the difference for semantic relevance was negligible (0.19 in-person and 0.18 remote, scale=0 to 1; d=0.14, p=0.19) as shown in FIG. 7, the difference for MATTR was small (0.75 in-person and 0.74 remote, scale=0 to 1; d=0.37, p<0.05) as shown in FIG. 8, and the difference for pronoun-to-noun ratio was moderate (0.48 in-person and 0.35 remote, scale=0 to 1; d=0.56, p<0.05) as shown in FIG. 9.

Results show that response length and semantic relevance of responses to the Cookie Theft picture description task are comparable under supervised, in-person and unsupervised, remote conditions. Small to moderate differences in vocabulary (MATTR) and pronoun-to-noun ratio may be due to differences in age.

### Example 3: Identifying Cognitive Impairment Using Digital Speech-Based Measures

Datasets from the Dementia Bank and Wisconsin Registry for Alzheimer's Prevention (WRAP) were evaluated for the first observation of Cookie Theft descriptions from 1011 healthy participants: 51 with MCI (based on MMSE scores) and 193 with dementia. From the samples, automatically extracted metrics were fit two classification models, which: (1) separated healthy from MCI participants; and (2) separated healthy from dementia participants. To avoid overfitting and spurious patterns in the data, k-fold cross-validation was used, the feature space was restricted to only clinically meaningful metrics, and simple logistic regression models were used with no more than 5 predictors.

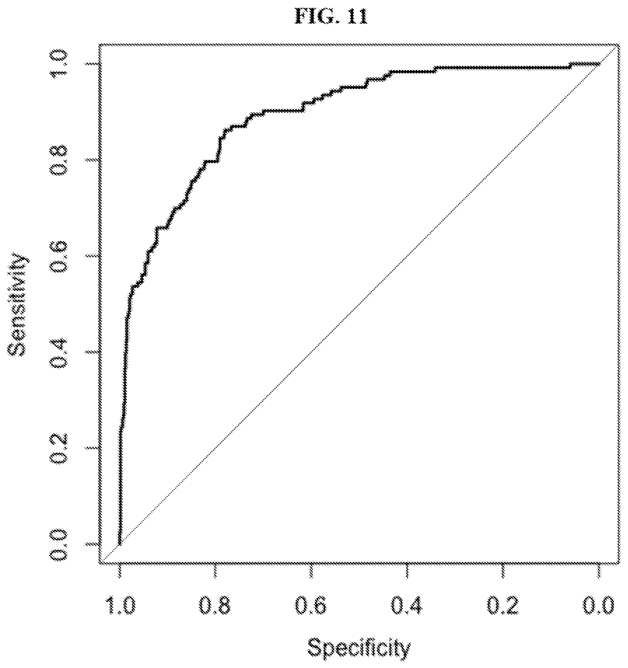

The MCI classifier separated healthy and MCI groups (ROC AUC=0.80; FIG. 10). Predictors were: MATTR (proportion of unique words on 10-word moving average; b=−16.0, z=−4.7, p-val<0.05); the ratio of pronouns to nouns (b=28.1, p-value<0.05), and propositional density (b=−15.0, p-value<0.05). The dementia classifier separated healthy and dementia groups (ROC AUC=0.89; FIG. 11). The predictors were: parse tree height (sentence complexity; b=−1.3, p-value<0.05), mean length of word (surface form; b=−3.9, p-val<0.05), the proportion of relevant details correctly identified in a picture description (b=10.7; p<0.05), type-to-token ratio (proportion of unique words; b=−11.0; p-value<0.05), and the duration of pauses relative to the total speaking duration (b=1.1, p-value=0.22).

Both classifiers separated groups with ROC AUC>=0.80, with the dementia classifier performing better than MCI classifier. Features in the models were clinically meaningful and interpretable, and relate to vocabulary (MATTR, propositional density, TTR, mean word length), syntax (parse tree height), language processing (pause duration/speaking duration), and ability to convey relevant picture details.

### Example 4: Evaluating Speech as a Prognostic Marker of Cognitive Decline

A study was performed to evaluate whether future cognitive impairment could be predicted from language samples obtained when speakers were still healthy. The study utilized features that were extracted from manual transcriptions of speech elicited from the Cookie Theft picture description task from (1) participants who were cognitively intact at time point 1 and remained cognitively intact at time point 2 and (2) participants who were cognitively intact at time point 1 and eventually developed mild cognitive impairment (MCI).

Two longitudinal data sets were used: the Wisconsin Registry for Alzheimer's Prevention and Dementia Bank. Participants were measured repeatedly, typically once per year. As part of the assessments, participants were recorded while completing the Cookie Theft picture description task (BDAE), and were assessed on the MMSE. Two groups of participants were identified: those who started as healthy participants and remained healthy throughout the study (non-decliners, N=795) and participants who started off as healthy and later became MCI (decliners, N=17). MCI was defined as those individuals who had MMSE scores between 24 and 26. Using transcripts of Cookie Theft recordings, metrics were extracted and used to classify between decliners and non-decliners using logistic regression. To minimize the chances of overfitting and spurious patterns in the data, the model was restricted to using only two clinically relevant features and model performance was evaluated using out-of-sample predictions with 2-fold cross-validation.

The results showed that there were two language features that could separate between decliners and non-decliners: the number of pauses throughout the recording (b=−6.0; p-value=0.12; although non-significant in the model, it substantially increased out-of-sample prediction accuracy) and the proportion of words spoken that were unique (b=0.43; p-value<0.05). Participants with more pauses and participants who used fewer unique words during the description were more likely to decline at a later time to MCI. The out-of-sample ROC AUC=0.71.

The results indicated that increased pausing between words and use of a smaller number of unique words were early harbingers of impending cognitive decline.

### Example 5: Automated Semantic Relevance as an Indicator of Cognitive Decline

The development and evaluation of an automatically extracted measure of cognition (semantic relevance) was performed using automated and manual transcripts of audio recordings from healthy and cognitively impaired participants describing the Cookie Theft picture from the Boston Diagnostic Aphasia Examination. The rationale and metric validation are described.

The measure of cognition was developed on one dataset and evaluated it on a large database (over 2000 samples) by comparing accuracy against a manually calculated metric and evaluating its clinical relevance.

The fully-automated measure was accurate (r=0.84), had moderate to good reliability (ICC=0.73), correlated with MMSE and improved the fit in the context of other automatic language features (r=0.65), and longitudinally declined with age and level of cognitive impairment.

This study demonstrates the use of a rigorous analytical and clinical framework for validating automatic measures of speech, and applied it to a measure that is accurate and clinically relevant.

The power of language analysis to reveal early and subtle changes in cognitive-linguistic function has been long recognized but challenging to implement clinically or at scale because of the time and human resources required to obtain robust language metrics. This is particularly true of picture description tasks, which are regarded as rich data sources because they require a broad range of cognitive and linguistic competencies to successfully complete. For example, the Boston Diagnostic Aphasia Examination (BDAE) includes an elicitation of The Cookie Theft picture description and this task is widely used clinically and in research across a swath of clinical conditions, including cognitive decline and dementia. The information extracted from transcripts of the picture descriptions provides insight to the likely sources of deficit and differential diagnosis. Yet, the burden of the analyses on human resources is prohibitively high for routine clinical use and impedes rapid dissemination of research findings. This study demonstrates the feasibility of using an automated algorithm to measure an interpretable and clinically relevant language feature extracted from picture descriptions while dramatically reducing the human burden of manually assigning codes to features of interest.

A commonly extracted, high-yield metric for the characterization of cognitive-linguistic function in the context of dementia involves assessment of the relationship of the words in the transcribed picture description to the word targets in the picture. This measure has been described with varying terminology, including “correct information units”, “content information units”, and “semantic unit idea density”. All these terms encapsulate essentially the same concept: the ratio of a pre-identified set of relevant content words to the total words spoken. For example, in the Cookie Theft picture description, people are expected to use the words “cookie,” “boy,” “stealing,” etc., corresponding to the salient aspects of the picture. An automated algorithm was developed to measure this relationship, called the Semantic Relevance (SemR) of participant speech. The Semantic Relevance metric provides a better frame for the measurement of this relationship. SemR measures the proportion of the spoken words that are directly related to the content of the picture, calculated as a ratio of related words to total words spoken. Like its manual predecessor, “semantic unit idea density”, the automated SemR metric provides an objective measure of the efficiency, accuracy, and completeness of a picture description relative to the target picture.

The goal of this study is two-fold. First, the process for measuring SemR is automated by transcribing recordings of picture descriptions using automatic speech recognition (ASR) and algorithmically computing SemR; done manually, this is a burdensome task and prohibitive at a large scale. Second, this study illustrates a rigorous analytical and clinical validation framework where SemR is validated on a new, large (over 2,000 observations) database. The study is organized in the following manner.

Analytical Validation: the first part of the study shows that the SemR scores remain accurate at each step that the measure is automated.
Section 1: Removing the human from the SemR computation. In this section, a large evaluation sample is used to show the accuracy achieved after automating the computation of SemR. This section first shows the accuracy achieved when the content units for calculating SemR are identified algorithmically rather than through manual coding. Second, this section shows the accuracy achieved when the transcripts are obtained through ASR instead of manually transcribing them.
Section 2: Removing the human from the data collection. An evaluation was conducted to determine what happens when the data collection is done remotely and without clinical supervision. To do this, a comparison was performed for the SemR scores between participants who provided picture descriptions in-clinic supervised by a clinician and at-home in an unsupervised setting.

Clinical Validation: the second part of the study demonstrates the relationship between SemR and cognitive function.
Section 3: Evaluation of the clinical relevance of SemR. The fully-automated version of SemR was evaluated for its clinical relevance computing its test-retest reliability, its association with cognitive function, its contribution to cognitive function above and beyond other automatically-obtained measures of language production, and its longitudinal change for participants with different levels of cognitive impairment.

### Methods

Development Dataset: A small data (25 participants, 584 descriptions of pictures) set was used for developing the SemR algorithm. These participants had amyotrophic lateral sclerosis (ALS) and frontotemporal dementia (FTD) of varying degrees of severity. The inclusion of participants with unimpaired speech along with speech impacted by dysarthria and cognitive impairment for developing the algorithm provided a rich data set with samples that varied in the picture descriptions' length and content.

Evaluation Dataset: The sources of the evaluation data included the Wisconsin Registry for Alzheimer's Prevention (WRAP) study, DementiaBank, and Amazon's Mechanical Turk. WRAP and DementiaBank conducted the data collection in-clinic with supervision from a clinician, and were evaluated for their degree of cognitive impairment. The data collection through Mechanical Turk was conducted remotely, where participants self-selected to participate in an online “speech task” study from their computers and were guided through the study via a computer application.

At each data collection, recorded descriptions of the Cookie Theft picture were obtained. The sample consisted of various characteristics, including participants who provided repeated measurements over the course of years, participants who completed a paired Mini Mental State Exam (MMSE), participants who provided the picture descriptions in-clinic supervised by a clinician, and participants who provided the picture descriptions from home. Additionally, the sample included transcripts that were manually transcribed, transcripts transcribed by ASR, and transcripts that were manually annotated by trained annotators to compute SemR. The WRAP participants were diagnosed according to a consensus conference review process as being cognitively unimpaired and stable over time (CU), cognitively unimpaired but showing a typical decline over time (CU-D), clinical mild cognitive impairment (MCI), and dementia (D). The DementiaBank participants were described as healthy controls (coded here as Cognitively Unimpaired [CU]) and as participants with Dementia. Mechanical Turk participants self-reported no cognitive impairment (CU), absent clinical confirmation. Table 1 shows descriptive statistics of the sample for each diagnostic group. Additionally, Table 2 shows the number of samples available for each type of data, for a total of 552 (DementiaBank), 2,186 (WRAP) and 595 (Mechanical Turk).

TABLE 1
Description of the evaluation sample

| | Evaluation Data | | | |
|---|---|---|---|---|
| Demographic | CU | CU-D | MCI | Dementia |
| Age Mean (SD) | 58.5 (10.5) | 63.6 (6.0) | 66.7 (6.4) | 71.2 (8.6) |
| Gender (% F) | 58% F | 61% F | 73% F | 65% F |
| Race (% Caucasian/White) | 93% W | 84% W | 78% W | 97% W |
| Education (% less than high | 1% < HS, | 2% < HS, | 12% < HS, | 33% < HS, |
| school, % completed high | 16% HS, | 10% HS, | 15% HS, | 31% HS, |
| school, % more than high | 83% > HS | 88% > HS | 73% > HS | 38% > HS |
| school) | | | | |
| Number of observations | 2,610 | 327 | 64 | 311 |
| Number of participants | 1,258 | 180 | 26 | 195 |

TABLE 2
Number of observations for each sample characteristic

| | Number of |
|---|---|
| Sample Characteristics | Observations |
| Speech was manually transcribed | 2,716 |
| Manual transcription was manually annotated to | 2,163 |
| manually calculate SemR | |
| Speech was transcribed using ASR | 2,921 |
| Speech was collected in-clinic | 2,716 |
| Speech was collected remotely | 595 |
| Speech sample was collected with paired MMSE | 2,564 |
| Speech was collected in close temporal proximity | 319 |
| (separated by approximately 1 week) | |

In the following sections, unless otherwise specified, each analysis used all the data that was available given the required characteristics (e.g., when estimating the accuracy of the automatically-computed SemR with the manually-annotated SemR, all observations where both sets of SemR scores were available were used for the analysis.)

### Development of Semantic Relevance

The automation of the SemR measure was developed because of the demonstrated clinical utility of picture description analysis, as well as its ability to provide insight into the nature of different deficit patterns and differential diagnosis. The goal of the SemR measure is to gauge retrieval abilities, ability to follow directions, and ability to stay on task in a goal-directed spontaneous speech task. The complex picture description task from the BDAE was used, where participants were shown a picture of a complex scene and were asked to describe it. SemR is higher when the picture description captures the content of the picture and is lower when the picture description shows signs of word finding difficulty, repetitive content, and overall lack of speech efficiency. In other words, SemR measures the proportion of the picture description that directly relates to the picture's content.

The algorithm operates as follows: First, the speech is transcribed. Then, each word is categorized according to whether it is an element from the picture or not. For this, the algorithm requires a set of inputs which indicate what elements from the picture need to be identified. For the Cookie Theft picture, we chose the 23 elements indicated in (e.g., boy, kitchen, cookie) and allowed the algorithm to accept synonyms (e.g., “young man” instead of “boy”). Finally, the total number of unique elements from the picture that a participant identifies is annotated and divided by the total number of words that the participant produced. Importantly, these keywords were fixed after development and were not modified during evaluation.

### ASR Transcription

Google Cloud's Speech-to-Text software transcribed the speech samples. The ASR algorithm was customized for the task at hand by boosting the standard algorithm such that the words that are expected in the transcript have increased probability that they would be correctly recognized and transcribed. This was implemented in Python using Google's Python application programming interface.

### Data Analysis

The data analysis is split into three sections to evaluate: 1) accuracy of the automatic algorithm, 2) sensitivity of SemR to the administration method, and 3) clinical utility of SemR by measuring differences in SemR scores across levels of cognitive impairment, and within-participant longitudinal change.

Section 1. Evaluation of Semantic Relevance: Removing the Human from the SemR Computation

In the manual implementation of SemR there are two steps that involve human intervention, including manually transcribing the participant's recorded picture description and then manually annotating the content units mentioned. To establish the analytical validity of the automated SemR, we tested replacement of human intervention in two ways. First, manual transcriptions were used to compare performance of the manually-annotated SemR to the algorithmically-computed SemR. Second, ASR-generated transcripts were used to compare the automatically-computed SemR scores with the manually-transcribed-and-annotated SemR and manually-transcribed-automatically-computed SemR scores. The goal of this series of analyses was to show that the automated accuracy was maintained relative to ground truth (human intervention) at each step of transcription and calculation of SemR.

To measure the accuracy achieved at each step, the correlation between each pair (using a mixed-effects model given the repeated measurements per participant) and the mean absolute error (MAE) of the two were computed.

Section 2. Evaluation of Semantic Relevance: Removing the Human from the Data Collection

Next, an evaluation was carried out on the feasibility of automating the data collection to be done remotely, without supervision, instead of in-clinic and supervised. A sample of 150 participants matched on age and gender was selected, half of whom provided data in-clinic (WRAP, DementiaBank) and half at-home (Mechanical Turk). The selected participants were only in-clinic participants who were deemed cognitively unimpaired by a clinician, and at-home participants who denied cognitive impairment. The final sample for this analysis consisted of 75 participants in-clinic and 75 participants at-home with average age 62 (SD=8.0) years old and with 42 F and 33 M in each group. A Welch's test (unequal variances) was conducted comparing the mean SemR scores of the two samples.

### Section 3. Evaluation of the Clinical Relevance of SemR

After establishing the accuracy and feasibility of fully automating the data collection and computation of SemR, an ASR transcript was generated and SemR for each participant was automatically computed. An evaluation of its clinical relevance was determined by: (a) estimating the test-retest reliability using intra-class correlation (ICC), standard error of measurement (SEM), and coefficient of variation (CV); (b) estimating its association with cognitive function and its contribution to cognitive function above and beyond other automatically-obtained measures of language production by fitting a model predicting MMSE and by classifying between disease groups (CU vs the three disease groups); and (C) estimating the longitudinal within-person change of SemR for participants at different levels of cognitive impairment using a growth curve model (GCM).

### Results

Section 1. Evaluation of Semantic Relevance: Removing the Human from the SemR Computation

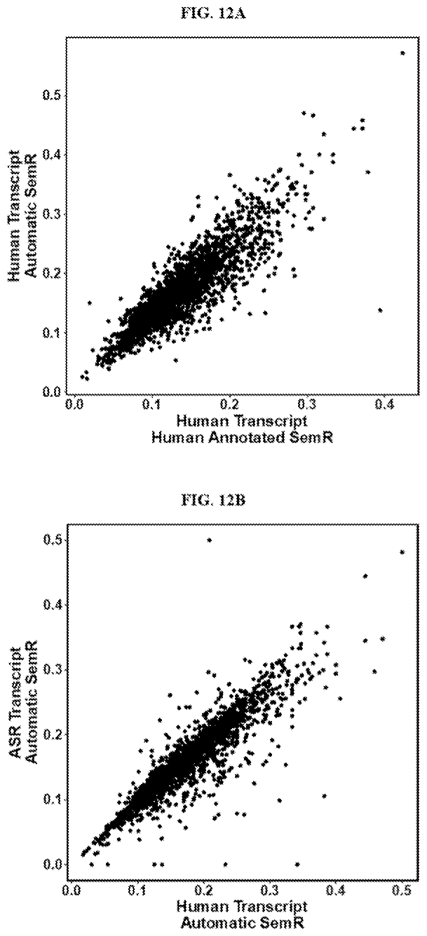

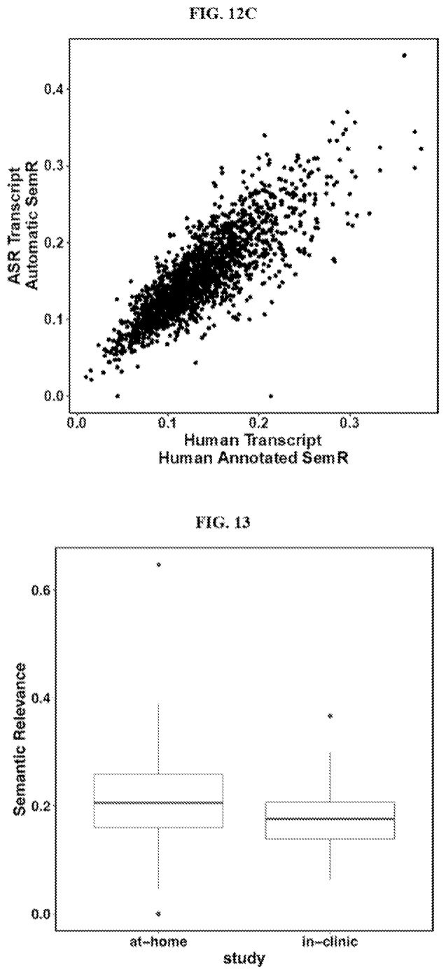

For the analytical validation of SemR, a comparison was carried out for the automatic SemR on manual transcripts, SemR calculated based on manual annotations on the manual transcripts, and automatic SemR on ASR transcripts. FIGS. 12A-12C show the plot for each comparison and Table 3 shows the correlations and mean absolute error (MAE). All three versions of SemR correlated strongly and had a small MAE, indicating that the automatic computation of SemR did not result in a substantial loss of accuracy.

TABLE 3
Correlations and differences between the manually-annotated,
manually-transcribed algorithmically-computed, and ASR-
transcribed algorithmically-computed SemR values

| Analysis | Correlation | MAE |
|---|---|---|
| Human-transcript-and-SemR vs | 0.87 | 0.04 |
| Human-transcript-automatic-SemR | | |
| Human-transcript-automatic-SemR vs | 0.95 | 0.01 |
| ASR-transcript-automatic-SemR | | |
| Human-transcript-and-SemR vs | 0.84 | 0.03 |
| ASR-transcript-automatic-SemR | | |

Section 2. Evaluation of Semantic Relevance: Removing the Human from the Data Collection

Next, the impact of the data collection method was evaluated by comparing SemR scores of supervised (in-clinic) and unsupervised (at-home) participants. A Welch's test indicated that the mean SemR scores were significantly different between the two groups (at home=0.21, in-clinic=0.18, t=2.55, p=0.01, Cohen's d=0.43). However, Cohen's d=0.43 indicated that the difference between the two groups was small. FIG. 13 shows the boxplots with the SemR scores for the at-home and in-clinic samples.

### Section 3. Evaluation of the Clinical Relevance of SemR

### Test-Retest Reliability

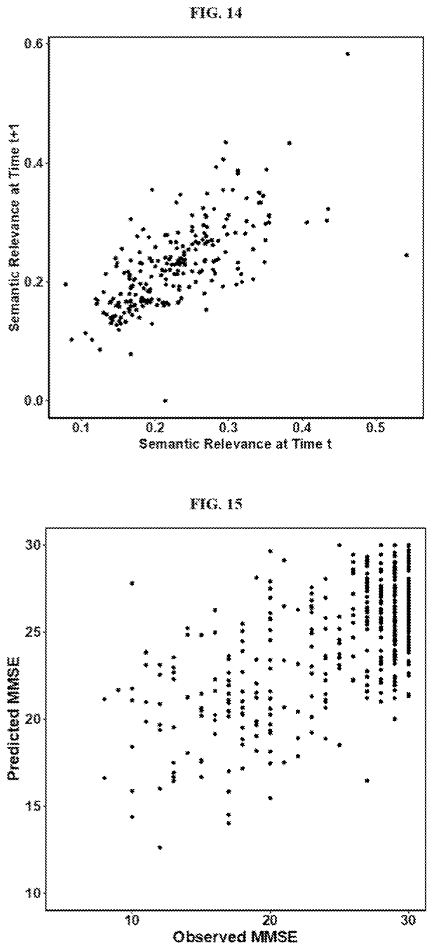

For evaluating the clinical validity of SemR, the test-retest reliability was first estimated. It was found that ICC=0.73, SEM=0.04, CV=19%. This was moderate to good reliability, which was considerably higher than most off-the-shelf language features extracted from text. FIG. 14 shows the test-retest plot.

Cross-Sectional Relationship between SemR and Cognitive Impairment

A series of models was generated to evaluate how SemR was related to cognitive impairment. The final results were the following. When using SemR alone, the correlation between SemR and MMSE was r=0.38. When using the set of automatically-computed language metrics (not including SemR), the correlation between the predicted and observed MMSE (using 10-fold cross-validation) was r=0.38 with MAE=4.4. Finally, when using SemR in addition to the set of metrics to predict MMSE, the correlation between the observed and predicted MMSE was r=0.65 and MAE=3.5. Finally, SemR's ability to classify disease (CUs vs the three clinical groups) above and beyond the MMSE alone was evaluated, and it was found that the AUC increased from AUC=0.78 (MMSE alone) to AUC=0.81 (MMSE and SemR). This indicated that SemR offered insight into one's cognition both as a stand-alone measure and above and beyond what was possible through other measures. FIG. 15 shows the observed and predicted MMSE scores for the final model.

### Longitudinal Trajectory of SemR

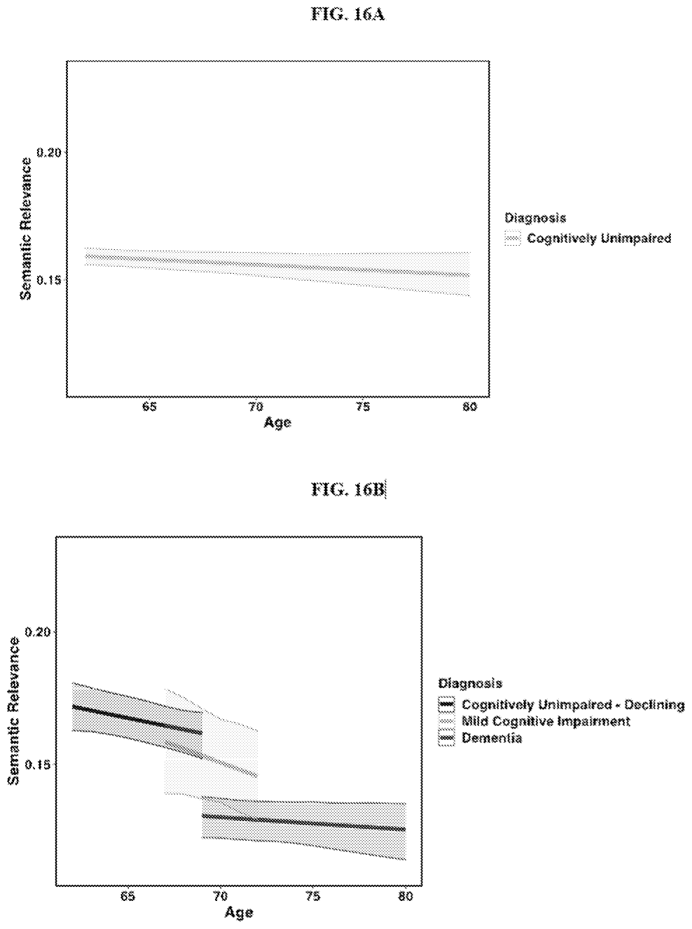

The longitudinal analyses showed that all groups had declining SemR scores. However, the CUs had slower-declining SemR scores than the impaired groups. Among the impaired groups, the results showed an apparent non-linear decline, where the scores started at the highest point among the CU-D participants, followed by the MCI participants with intermediate scores and the steepest decline, finally followed by the dementia participants, who had the lowest SemR scores and whose trajectory flattened again. Table 4 shows GCM parameters for the four groups. FIG. 16A and FIG. 16B show the expected longitudinal trajectories according to the GCM parameters for the healthy (a) and cognitively impaired (b) groups. Although all data was used for the analyses, for easier visualization of the results in the cognitively impaired groups the plots were restricted to the age range with the greatest density of participants in each group (approximately between Q1 and Q3 for each cognition group).

TABLE 4
Parameter estimates for the GCMs for each cognitive group

| | CU Estimate | | CU-D Estimate | | MCI Estimate | | Dem Estimate | |
|---|---|---|---|---|---|---|---|---|
| Parameter | (S.E.) | | (S.E.) | | (S.E.) | | (S.E.) | |
| Fixed effects | | | | | | | | |
| Intercept (centered at | 0.158 | (.002) | 0.167 | (.004) | .163 | (.01) | .132 | (.005) |
| age 65) | | | | | | | | |
| Slope | −.0004 | (.0002) | −.0014 | (.0006) | −.0026 | (.0015) | −.0005 | (.0004) |

| | | | | |
|---|---|---|---|---|
| Random effects | | | | |
| Participant intercepts | 0.03 | 0.05 | 0.03 | 0.03 |
| SD | | | | |
| Residuals SD | 0.04 | 0.04 | 0.04 | 0.05 |

### Discussion

The present study builds on the work of Mueller and colleagues (2018) which evaluated the contribution of connected language, including the Cookie Theft picture descriptions, to provide early evidence of mild cognitive-linguistic decline in a large cohort of participants. They used latent factor analysis to discover that longitudinal changes in the “semantic category” of measures were most associated with cognitive decline. Semantic relevance in this highly structured picture description task, captures the ability to speak coherently by maintaining focus on the topic at hand. Some studies have shown that older adults tend to produce less global coherence (and more irrelevant information) in discourse than younger adults. Furthermore, more marked discourse coherence deficits have been reported across a variety of dementia types including Alzheimer's disease dementia and the behavioral variant of frontotemporal dementia. The neural correlates of coherence measures are difficult to capture, since multiple cognitive processes contribute to successful, coherent language. However, the SemR measure is an ideal target for the cognitive processes known to be affected across stages of dementia. For example, in the case of Alzheimer's disease dementia, lower semantic relevance could be the result of a semantic storage deficit, search and retrieval of target words or inhibitory control deficits, all of which can map onto brain regions associated with patterns of early Alzheimer's disease neuropathology.

The development of the automated SemR metric in the present report was intended to mitigate the labor-intensive task of coding content units manually, in order to validate a tool that can expedite research and enhance clinical assessment in the context of pre-clinical detection of cognitive decline. The clinical validation of SemR yielded results that were consistent with previous research (e.g., declining scores for older and more cognitively impaired participants).

In addition to developing and thoroughly evaluating the automatically-extracted language measure SemR, this article illustrates the use of a rigorous framework for analytical and clinical validation for language features. There has been a great deal of recent interest in automated analysis of patient speech for assessment of neurological disorders. In general, machine learning (ML) is often used to find “information” in this high-velocity data stream by transforming the raw speech samples into high-dimensional feature vectors that range from hundreds to thousands in number. The assumption is that these features contain the complex information relevant for answering the clinical question of interest. However, this approach carries several risks and most measures of this type fail to undergo rigorous validation, both because large datasets containing speech from clinical groups are difficult to obtain, and because there is no way to measure the accuracy of an uninterpretable feature, for which there is no ground truth. The consequence is measures that vary widely in their ability to capture clinically-relevant changes. In contrast, the best practices for “fit for purpose” algorithm development, as set forth by the Digital Medicine Society, were followed herein. First, the algorithm was developed on one set of data from participants with ALS and FTD, and then tested on a separate, large, out-of-sample data set from a different clinical population (cognitively unimpaired, MCI, and dementia), thus fully separating the development, freezing of the algorithm, and testing. During the testing of the algorithm, the method for evaluating accuracy at each step of the automation was shown. Finally, SemR was validated as a clinical tool, where an evaluation was performed on its reliability, association with cognitive function, and change over time.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A device for evaluating cognitive function based on speech, the device comprising:
audio input circuitry configured to receive an audio signal provided by a subject;
signal processing circuitry configured to:
process the input signal to detect one or more metrics of speech of the subject; and
analyze the one or more metrics of speech using a speech assessment algorithm to generate an evaluation of a cognitive function of the subject.

2. The device of claim 1, wherein the evaluation of the cognitive function comprises detection or prediction of future cognitive decline.

3. The device of claim 1, wherein the evaluation of the cognitive function comprises a prediction or classification of normal cognition, early mild cognitive impairment, mild cognitive impairment, or dementia.

4. The device of claim 1, wherein the one or more metrics of speech of the subject comprises a metric of semantic relevance, word count, ratio of unique words to total number of words (MATTR), pronoun-to-noun ratio, propositional density, number of pauses during an audio speech recording within the input signal, or any combination thereof.

5. The device of claim 4, wherein the metric of semantic relevance measures a degree of overlap between a content of a picture and a description of the picture detected from the speech in the input signal.

6. The device of claim 1, wherein the signal processing circuitry is further configured to display an output comprising the evaluation.

7. The device of claim 1, wherein the notification element comprises a display.

8. The device of claim 7, wherein the signal processing circuitry is further configured to cause the display to prompt the subject to provide a speech sample from which the input signal is derived.

9. The device of claim 1, wherein the signal processing circuitry is further configured to utilize at least one machine learning classifier to generate the evaluation of the cognitive function of the subject.

10. The device of claim 9, wherein the signal processing circuitry is configured to utilize a plurality of machine learning classifiers comprising a first classifier configured to evaluate the subject for a first cognitive function or condition and a second classifier configured to evaluate the subject for a second cognitive function or condition.

11. A computer-implemented method for evaluating cognitive function based on speech, the method comprising:
receiving, with audio input circuitry, an input signal provided by a subject;
processing, with signal processing circuitry, the input signal to detect one or more metrics of speech of the subject; and
analyzing, with signal processing circuitry, the one or more metrics of speech using a speech assessment algorithm to generate an evaluation of a cognitive function of the subject.

12. The method of claim 11, wherein the evaluation of the cognitive function comprises detection or prediction of future cognitive decline.

13. The method of claim 11, wherein the evaluation of the cognitive function comprises a prediction or classification of normal cognition, early mild cognitive impairment, mild cognitive impairment, or dementia.

14. The method of claim 11, wherein the one or more metrics of speech of the subject comprises a metric of semantic relevance, word count, ratio of unique words to total number of words (MATTR), pronoun-to-noun ratio, propositional density, number of pauses during an audio speech recording within the input signal, or any combination thereof.

15. The method of claim 14, wherein the metric of semantic relevance measures a degree of overlap between a content of a picture and a description of the picture detected from the speech in the input signal.

16. The method of claim 11, wherein the signal processing circuitry is further configured to display an output comprising the evaluation.

17. The method of claim 11, wherein the notification element comprises a display.

18. The method of claim 11, further comprising prompting the subject to provide a speech sample from which the input signal is derived.

19. The method of claim 11, comprising utilizing at least one machine learning classifier to generate the evaluation of the cognitive function of the subject.

20. The method of claim 19, wherein the at least one machine learning classifier comprises a first classifier configured to evaluate the subject for a first cognitive function or condition and a second classifier configured to evaluate the subject for a second cognitive function or condition.

21. A computer-implemented method for generating a speech assessment algorithm comprising a machine learning predictive model for evaluating cognitive function based on speech, the method comprising:
receiving input signal comprising speech audio for a plurality of subjects;
processing the input signal to detect one or more metrics of speech in the speech audio for the plurality of subjects;
identifying classifications corresponding to cognitive function for the speech audio for the plurality of subjects; and
training a model using machine learning based on a training data set comprising the one or more metrics of speech and the classifications identified in the speech audio, thereby generating a machine learning predictive model configured to generate an evaluation of cognitive function based on speech.

22. The method of claim 21, wherein the evaluation of the cognitive function comprises detection or prediction of future cognitive decline.

23. The method of claim 21, wherein the evaluation of the cognitive function comprises a prediction or classification of normal cognition, early mild cognitive impairment, mild cognitive impairment, or dementia.

24. The method of claim 21, wherein the one or more metrics of speech of the subject comprises a metric of semantic relevance, word count, ratio of unique words to total number of words (MATTR), pronoun-to-noun ratio, propositional density, number of pauses during an audio speech recording within the input signal, or any combination thereof.

25. The method of claim 24, wherein the metric of semantic relevance measures a degree of overlap between a content of a picture and a description of the picture detected from the speech in the input signal.

26. The method of claim 21, further comprising configuring a computing device with executable instructions for analyzing the one or more metrics of speech using the machine learning predictive model to generate an evaluation of a cognitive function of a subject based on the input speech sample.

27. The method of claim 26, wherein the computing device is configured to display an output comprising the evaluation.

28. The method of claim 26, wherein the computing device is a desktop computer, a laptop, a smartphone, a tablet, or a smartwatch.

29. The method of claim 26, wherein the configuring the computing device with executable instructions comprises providing a software application for installation on the computing device.

30. The method of claim 29, wherein the computing device is a smartphone, a tablet, or a smartwatch; and wherein the software application is a mobile application.

31. The method of claim 30, wherein the mobile application is configured to prompt the subject to provide the input speech sample.

32. The method of claim 21, wherein the input speech sample is processed by one or more machine learning models to generate the one or more metrics of speech; wherein the machine learning predictive model is configured to the evaluation of cognitive function as a composite metric based on the one or more metrics of speech.
